(19) United States
(12) Patent Application Publication
(10) Pub. No.: US 2025/0205274 A1
(43) Pub. Date: Jun. 26, 2025

(54) FORMULATIONS FOR TREATMENT OF XEROSTOMIA, AND METHODS THEREFOR

(71) Applicant: Med1 Ventures, LLC, Raleigh, NC (US)

(72) Inventors: Howard Ketelson, Dallas, TX (US); Joseph Huber, Raleigh, NC (US); Steven Bacich, Half Moon Bay, CA (US); Timothy Willis, Raleigh, NC (US)

(21) Appl. No.: 18/966,350

(22) Filed: Dec. 3, 2024

Related U.S. Application Data
(60) Provisional application No. 63/612,650, filed on Dec. 20, 2023.

(51) Int. Cl. A61K 31/728; A61K 9/00; A61K 45/06

(52) U.S. Cl. CPC A61K 31/728; A61K 9/0053; A61K 45/06

(57) ABSTRACT

An oral or nasopharynx product that provides long-lasting relief and treatment of dry mouth, known medically as xerostomia. A formulation described herein provides a durable relief by comprising a mucoadhesive polymer modified to bind and anchor to saliva in a mouth and back of the throat. The formulation firmly binds to a mucin layer in the mouth due to anchoring groups' binding capabilities. Useful mucoadhesive polymer includes medium to high molecular weight hyaluronic acid. Useful anchoring group includes boronic acid. The formulation then comprises a salivary enhancer, such as xylitol. The formulation can be provided in an aqueous or solid medium that helps to maintain a near-neutral pH condition. The formulation may further comprise flavoring agents, sweeteners, surfactants, foaming agents, and so on. The disclosure also provides for methods of alleviating the symptoms of dry mouth, methods for delivering pharmaceuticals, and methods of manufacture of the formulations.

### TECHNICAL FIELD

The present application generally relates to compositions for treatment of xerostomia or dry mouth and related disorders, and more specifically, to oral compositions, formulations, gums, pastes, sprays, lozenges, or mouthwashes comprising polymers modified with functional groups (termed “adherent polymers”) that can bind either covalently, physically, or electrostatically, or a combination thereof, with oral mucosal and dental surfaces for extended periods of time. The present application also applies to the surfaces of the nasopharynx and nasal passages affected by xerostomia and is treatable by sprays, mists, and drops.

### BACKGROUND

Xerostomia, or dry mouth, is a universal problem that can affect anybody, regardless of sex, race, age, or other conditions and has a variety of causes James Guggenheimer, Paul A. Moore, Xerostomia: Etiology, Recognition and Treatment, The Journal of the American Dental Association, Volume 134, Issue 1, 2003, Pages 61-69. However, it is more frequent in women than in men, mainly due to the reduction of hormones in perimenopause, in the same manner as dryness appears in other mucosa such as the vagina and the eyes. The most common disease causing xerostomia is Sjögren's syndrome, that occurs predominantly in postmenopausal women. Approximately 3% of Americans suffer from Sjögren's syndrome, with 90% of these patients being women with a mean age at diagnosis of 50 years. Sjögren's syndrome is characterized by lymphocytic infiltration of salivary and lacrimal glands, resulting in xerostomia and xerophthalmia. This combination is called the sicca complex. At the present time there is no known cure for the disease and current therapies merely manage symptoms such as using over the counter products. Cathy L. Bartels, Pharm.D., Assistant Professor, Pharmacy Practice, School of Pharmacy and Allied Health Sciences, University of Montana, https://oralcancerfoundation.org/complications/xerostomia/. One out of every four adults suffers from dry mouth. Agostini B A, Cericato G O, Silveira E R D, Nascimento G G, Costa F D S, Thomson W M, Demarco F F. How Common is Dry Mouth? Systematic Review and Meta-Regression Analysis of Prevalence Estimates. Braz Dent J. 2018 November-December; 29 (6): 606-618. Furthermore, dry mouth associated with multiple systemic and psychiatric diseases, such as diabetes, cancer, anxiety, depression, allergies, autoimmune diseases, stress, alcoholism, and the like. Also, it appears as a secondary effect to multiple treatments of these and other diseases that are becoming more frequent, even reaching epidemic levels.

The etiology of xerostomia is complex and difficult to diagnose in that it can be silent in many cases and may not show symptoms until it is very advanced. An estimated 50% of xerostomia patients do not show any symptoms, and what is medically more relevant, up to 50% of salivary flow may be lost without yet perceiving dryness in the mouth or before xerostomia manifests itself with signs or symptoms. Dryness in the mouth also can manifest in dry nasal passages and throat that can create burning sensation, hoarseness, disrupt sleep, and the quality of life.

Xerostomia affects both people with dentition (toothed patients) and toothless patients. The consequences of xerostomia in the first group are heavily manifested, since an alteration of the natural buffering capacity of saliva also occurs a few months after syndrome instauration, due to the deficit in salivary flow, leading to short-term structural deterioration of the hard tissues in the mouth (teeth). Su N, Marek C L, Ching V, Grushka M. Caries Prevention for Patients with Dry Mouth. J Can Dent Assoc. 2011; 77: b85. PMID: 21774875.

In toothed patients with xerostomia, the saliva that under normal physiological conditions acts as a defense, barrier, and reinforcement, is no longer effective or sufficient to slow down the demineralization of enamel and dentin caused by daily aggressions (cariogenic bacteria, acids, drops in pH, dental bruxism, or clenching). Thus, rapidly evolving caries and atypical caries, such as neck caries, occur as a result of the postprandial (after eating) decrease in oral pH, which can damage or completely destroy teeth within a few months. Furthermore, a series of symptoms and signs can occur independently of whether there are teeth or not, which can alter the patient's quality of life.

In toothless patients, xerostomia does not cause dental destruction since the patient has already lost all their teeth. However, the appearance of signs and symptoms can be even greater than for the first group, with effects such as lingual fissures, viscous saliva, and buccal sores.

Different types of glands are responsible for the secretion of saliva. Mucilaginous saliva is secreted by the minor salivary glands (labial, palatine, lingual), whereas aqueous saliva is secreted by the parotid and submaxillary glands in much larger amounts.

Saliva contains various types of mucins, which are a family of glycoproteins that play a crucial role in lubricating and protecting the oral cavity. Sialic acid is an important moiety attached along the backbone of mucins and has a variety of important roles, such as radical scavenging (see, for example: Yuki Ogasawara, Tomoyuki Namai, Fumihiko Yoshino, Masaichi-Chang-il Lee, Kazuyuki Ishii, Sialic Acid is an Essential Moiety of Mucin as a Hydroxyl Radical Scavenger, FEBS Letters, Volume 581, Issue 13, 2007, Pages 2473-2477).

The specific types of mucins present in saliva include: Tabak, L. A., Levine, M. J., Mandel, I. D. and Ellison, S. A. (1982), Role of salivary mucins in the protection of the oral cavity. Journal of Oral Pathology & Medicine, 11:1-17.

MUC5B: This is the predominant mucin in saliva. It forms a gel-like network that provides viscosity and lubrication to the mouth, throat, and esophagus. MUC5B helps in swallowing and forms a protective barrier on the oral mucosa.

MUC7: This mucin is found in both saliva and the mucus layer that lines the oral cavity. MUC7 exhibits antimicrobial properties and can help prevent the growth of certain bacteria in the mouth. It also contributes to the lubrication of the oral tissues.

MUC1, MUC4, MUC2, and others: While MUC5B and MUC7 are the most well-studied mucins in saliva, other mucins, such as MUC1, MUC4, and MUC2, have also been detected in smaller amounts. These mucins may have additional roles in oral health and lubrication.

The composition and levels of mucins in saliva can vary among individuals and can be influenced by factors such as genetics, oral health, and overall health. The presence of these mucins in saliva helps maintain oral health by providing lubrication, protection against pathogens, and contributing to the integrity of the oral mucosa.

Dry mouth symptoms are frequently present in patients whose salivary secretion, both stimulated and unstimulated, is normal. In contrast, there are patients with true hyposalivation who do not complain of buccal dryness. Perception by the patient largely depends on the salivary component that is lacking. Buccal dryness is felt when mucilaginous components are missing (scientifically called resting or unstimulated saliva), which lubricate and provide comfort, although the amount of salivary flow (flow of aqueous or stimulated saliva) may be the same. In contrast, patients with a decrease in salivary flow may not perceive dryness if they preserve mucilaginous saliva. There is great individual variability regarding salivary flow and can differ by up to 50% patient to patient.

Xerostomia can make to varying degrees, talking, chewing, and swallowing difficult, and also oral hygiene, since it favors the accumulation of dental plaque because the salivary flow no longer washes out bacteria. Furthermore, it reduces the buffer effect of saliva, making the oral pH become acidic, destroying the teeth. It is related to many chronic pharyngitis, mucositis, and even to digestive disorders. At a local level it can cause a feeling of burning and/or pain in the soft tissues and in the tongue, a need for moisturizing the mouth at short time intervals, leaves surfaces of the teeth rough, a sliver of saliva between the teeth or the tongue and the palate, a feeling of pressure or tightness in a group of teeth, and even pain. It causes a fast progression of tooth decay, it facilitates periodontal diseases (gingivitis), and it wears out teeth due to greater abrasion and friction without lubrication between dental surfaces. It also causes fissures in the lips and bacterial infections (candidiasis, sialadenitis), halitosis, and it can even produce eating disorders, insomnia, irritability, or depression. Likewise, xerostomia affects the sufferer's quality of life from the point of view of sociability: there is a lack of interest for eating in company, for going out, or for talking in groups.

It is estimated that more than 500 families of drugs have xerostomia as a side effect, which is one of the main reasons for the discontinuation of medication by patients. These are responsible for most cases of xerostomia. Salivary deficiency usually lasts a long time after an extended treatment, despite drug discontinuation. Some exemplary drugs that are known to produce this effect include but are not limited to, diuretics (hydrochlorothiazide, amiloride), sedatives, antidepressants (serotonin reuptake inhibitors and especially tricyclic antidepressants), antihypertensives, anti-inflammatory drugs, decongestants (phenylpropanolamine, pseudoephedrine), anxiolytic drugs (diazepam), anticholinergic-type anti-spasm drugs (atropine, oxybutynin), antidiarrheal (loperamide, diphenoxylate), antihistamines (chlorphenamine, loratadine), non-steroid anti-inflammatory drugs (piroxicam, ibuprofen), opioid analgesics (morphine), muscle relaxants (baclofen), bronchodilators (ipratropium, salbutamol), anti-Parkinson drugs (levodopa, biperiden), antiacne drugs (isotretinoin), and antipsychotics such as phenothiazines and butyrophenones. Oncological treatments such as radiotherapy (one of the most widely recognized causes) and chemotherapy also often result in xerostomia. Further, consumption of addictive substances such as alcohol, tobacco, and drugs also causes xerostomia.

Thus, it can be seen that xerostomia is a very complex syndrome, and it is important to diagnose and treat it so that it does not seriously affect the patient's health and compromise the quality of life.

Until now, there have been limited successful attempts regarding treatment focused on using a variety of formulations imitating natural saliva to try to substitute the absent natural saliva or produce saliva. Radzki D, Wilhelm-Węglarz M, Pruska K, Kusiak A, Ordyniec-Kwaśnica I. A Fresh Look at Mouthwashes-What Is Inside and What Is It For? Int J Environ Res Public Health. 2022 Mar. 25; 19 (7): 3926. Xylitol is used in a number of dry mouth products as a sweetener to enhance salivary flow, but its effect is transitory as it does not last longer than a few hours and requires repeated dosing. Sugars such as xylitol or sorbitol do not act as a lubricant or provide protection against tissue desiccation. Mucoadhesive polymers such as hyaluronic acid has been used for various purposes in dry mouth applications. For example, EP2745836B1, US20220401321A1, U.S. Pat. No. 11,324,681B2, U.S. Pat. No. 11,400,035B2, and U.S. Pat. No. 7,195,753B1 disclose the use of hyaluronic acid in various types of formulations to address situations such as oral diseases, bad breath and the like. However, like many mucoadhesive polymers, its effect is only transient as its binding in the mouth is challenging to be long-lasting due to the continuous mechanical and saliva turnover events that happen within the oral cavity.

The vast majority of oral rinses that are currently available require multiple rinses throughout the day as the key ingredients, such as lubricants and saliva enhancers, are washed away as a result of limited contact time and effective binding to the oral mucosa. Exposure to topically administered oral formulations is typically in the range of a few hours maximum. Extended exposure has been achieved by the use of highly viscous formulations, but these are difficult to use in mouth rinse applications and also have limited retention due to the reliance on physical binding to the mucosa, which only provides temporary relief and oral protection. Therefore, there is still a need for providing alternative compositions for treating xerostomia that extends the duration to protect, hydrate, lubricate, and reduce pain associated with inflammation, enhance salivary flow for longer durations, as well as improve the quality of life of affected persons.

In view of the foregoing, it is an object of this application to provide formulations that have at least the following properties:
(1) Moisturizing properties: Dry mouth rinses are formulated to help mimic the natural lubricating and cleansing action of saliva. They contain ingredients that provide lubrication and moisture to the mouth, throat, and oral tissues.
(2) Saliva stimulation: Some dry mouth rinses may also contain substances that help stimulate saliva production. These substances, such as xylitol, sorbitol, erythritol, peptides, or enzymes, can encourage the natural flow of saliva and provide additional oral protection.
(3) Soothing effect: Dry mouth rinses often have soothing properties that can help relieve dry mouth symptoms, including a dry or sore throat or nasopharynx, difficulty swallowing, pain or a sticky sensation in the mouth.
(4) Oral health benefits: Dry mouth can increase the risk of dental problems, such as tooth decay and gum disease, due to reduced saliva's protective and cleansing effects. Dry mouth rinses often contain fluoride or antimicrobial agents to help prevent tooth decay and reduce the growth of bacteria in the mouth.
(5) Alcohol-free and gentle: Many dry mouth rinses are alcohol-free to prevent further drying of the oral tissues. They are typically formulated to be gentle and mild, suitable for individuals with sensitive mouths.
(6) Dry mouth rinses are also formulated to reduce the number of rinse applications required per day. These would then enhance patient compliance and also offer more convenience to improved efficacy benefits. This requires that the exposure of the active ingredient(s) is maintained over a therapeutic threshold over several hours, preferably all day.

To further enhance oral mucosa retention time, and hence enhance exposure time, inventors have surprisingly discovered that an adherent lubricant can chemically bind to mucins in saliva. Thus, the present application relates to the use of an adherent polymer to chemically react with mucins in the mouth and also deliver functional additives to prolong salivary production and reduce pain and inflammation. The adherent polymer is a mucoadhesive polymer modified with a mucin binding group along with the backbone of the polymer.

The formulation further includes one or more chemical or biologic pharmaceutically active agents and cannabinoids and is selected from a group comprising steroids, cooling agents, lubricants, anti-hypertensive agents, mucin secretagogues, anti-microbial agents, pain-relieving agents, anti-inflammatory agents, and combinations thereof. Vasudevan K, Stahl V. Cannabinoids Infused Mouthwash Products are as Effective as Chlorhexidine on Inhibition of Total-Culturable Bacterial Content in Dental Plaque Samples. J Cannabis Res. 2020 Jun. 23; 2 (1): 20.

The formulation can be in many product forms but preferably a solution such as a rinse or spray or lozenge. Oral or nasopharynx rinses or sprays work by providing moisture and lubrication to the oral cavity or nasopharynx, helping to alleviate the discomfort and potential oral, dental, or mucosal health problems associated with dry mouth.

The formulations described herein have several uses. The present application provides, for example, methods of treating a patient suffering from xerostomia or its related complications manifested from metabolic or genetic conditions or disorders, metabolic diseases, chronic diseases or disorders, neurodegenerative disorders, metabolic conditions, hepatology, cancer, or respiratory, hematological, orthopedic, cardiovascular, renal, skin, vascular, or ocular complications.

### SUMMARY

Embodiments disclosed herein include compositions for treatment of xerostomia or dry mouth and related disorders, and more specifically, to oral compositions, formulations, gums, pastes, sprays, lozenges, or mouthwashes comprising polymers modified with functional groups (termed “adherent polymers”) that can bind either covalently, physically, or electrostatically, or a combination thereof, with oral mucosal and dental surfaces for extended periods of time. The formulation is applied to the mouth and binds to the existing saliva. The formulation includes a mucoadhesive polymer, an anchoring group and a saliva enhancer binds to the existing saliva and extends the saliva time in the mouth. A variety of delivery modalities are available, including, but not limited to, oral rinse, lozenge, and spray.

### BRIEF DESCRIPTION OF FIGURE

FIG. 1 is a graph illustrating normalized concentration of HA control and AX01-04 in sampled eluate over time.

### DETAILED DESCRIPTION

The definitions provided herein are to facilitate understanding of certain terms used frequently herein and are not meant to limit the scope of the present disclosure.

As used in this specification and the appended claims, the singular forms “a,” “an,” and “the” encompass embodiments having plural referents unless the content clearly dictates otherwise.

Unless otherwise indicated, all numbers expressing feature sizes, amounts, and physical properties used in the specification and claims are to be understood as being modified in all instances by the term “about.” Accordingly, unless indicated to the contrary, the numerical parameters set forth in the foregoing specification and attached claims are approximations that can vary depending upon the desired properties sought to be obtained by those skilled in the art utilizing the teachings disclosed herein.

As used in this specification and the appended claims, the term “or” is generally employed in its sense including “and/or” unless the content clearly dictates otherwise.

The phrase “pharmaceutically acceptable carrier” is used herein to include, for example, pharmaceutically acceptable materials, compositions or vehicles, such as a liquid or solid filler, diluent, solvent, or encapsulating material involved in carrying or transporting any subject composition from one organ or portion of the body to another organ or portion of the body. Each carrier must be “acceptable” in the sense of being compatible with the other ingredients of a subject composition and not injurious to the patient. Exemplary materials that may serve as pharmaceutically acceptable carriers include, but not limited to: sugars, such as lactose, glucose, and sucrose; malt; starches, such as corn starch and potato starch; polysaccharides and their and their derivatives, such as cellulose, sodium carboxymethyl cellulose, hydroxyethyl cellulose, methyl and ethyl cellulose, cellulose acetate, agar, alginic acid, sodium alginate, gum tragacanth, gum arabic, gum Karaya, carrageenan, xanthan gum; gelatin; talc; oils, such as castor oil, mineral oil, peanut oil, cottonseed oil, sunflower oil, sesame oil, olive oil, corn oil and soybean oil; alcohols such as ethyl alcohol; glycols, such as propylene glycol; polyols, such as glycerin, sorbitol, mannitol and polyethylene glycol; esters, such as ethyl oleate and ethyl laurate; buffering agents, such as magnesium hydroxide and aluminum hydroxide; pyrogen-free water; isotonic saline; Ringer's solution; phosphate buffer solutions; and the like.

The term “prophylactic or therapeutic” treatment is used herein to include administration to the host of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if it is administered after manifestation of the unwanted condition, the treatment is therapeutic, (i.e., it is intended to diminish, ameliorate, or stabilize the existing unwanted condition or side effects thereof).

The phrase “therapeutically effective amount” is used herein to refer to an amount of a formulation or composition disclosed herein that produces some desired effect at a reasonable benefit/risk ratio applicable to any medical treatment. In certain embodiments, the phrase refers to the amount necessary or sufficient to eliminate or reduce medical symptoms for a period of time. The effective amount may vary depending on such factors as the disease or condition being treated, the particular targeted constructs being administered, the size of the subject, or the severity of the disease or condition. One of ordinary skill in the art may empirically determine the effective amount of a particular composition without necessitating undue experimentation.

In certain embodiments, the pharmaceutical compositions described herein are formulated in a manner such that the compositions will be delivered to a patient in a therapeutically effective amount, as part of a prophylactic or therapeutic treatment. The desired amount of the composition to be administered to a patient will depend on absorption, inactivation, and excretion rates of the drug as well as the delivery rate of the formulations and compositions from the subject compositions. It is to be noted that dosage values may also vary with the severity of the condition to be alleviated. It is to be further understood that for any particular subject, specific dosage regimens should be adjusted over time according to the individual need and the professional judgment of the person administering or supervising the administration of the compositions. Typically, dosing will be determined using techniques known to one skilled in the art.

Additionally, the optimal concentration and/or quantities or amounts of any particular formulation or composition may be adjusted to accommodate variations in the treatment parameters. Such treatment parameters include the clinical use to which the preparation is put, e.g., the site treated, the type of patient, e.g., human or non-human, adult or child, and the nature of the disease or condition. In certain embodiments, the dosage of the subject compositions provided herein may be determined by reference to the plasma concentrations of the therapeutic composition or other encapsulated materials. For example, the maximum plasma concentration (Cmax) and the area under the plasma concentration-time curve from time 0 to infinity may be used.

When used with respect to a pharmaceutical composition or other material, the term “sustained release” is art-recognized. For example, a subject composition which releases a substance over time may exhibit sustained release characteristics, in contrast to a bolus type administration in which the entire amount of the substance is made biologically available at one time. For example, in particular embodiments, upon contact with body fluids including blood, spinal fluid, mucus secretions, lymph or the like, one or more of the pharmaceutically acceptable excipients may undergo gradual or delayed degradation (e.g., through hydrolysis) with concomitant release of any material incorporated therein, e.g., a therapeutic and/or biologically active solvate or hydrate and/or composition, for a sustained or extended period (as compared to the release from a bolus). This release may result in prolonged delivery of therapeutically effective amounts of any of the therapeutic agents disclosed herein.

The phrases “systemic administration,” “administered systemically,” “peripheral administration” and “administered peripherally” are used herein to include the administration of a subject composition, therapeutic, or other material at a site remote from the disease being treated. Administration of an agent for the disease being treated, even if the agent is subsequently distributed systemically, may be termed “local” or “topical” or “regional” administration, other than directly into the central nervous system, e.g., by subcutaneous administration, such that it enters the patient's system and, thus, is subject to metabolism and other like processes.

Most dry mouth products use a viscosity enhancer for enhance retention and also provide some comfort as the polymer will form a film on the lining of the mouth cavity. This type of formulation approach uses rheology to dominate via the formation of a thick film when the mouth product is introduced in the mouth. Following rinsing, there will be some form of shear in the mouth and also dilution with saliva production. For thin films, the lubrication profile of the polymer, which impacts slipperiness and friction reduction, is important. The present application also describes dry mouth polymer has both viscosity and lubrication function and aims to optimize both mouth feel and retention on the target substrates. The use of an adherent polymer to form a lubricating film on application followed by reduction to a thin substantive film is the basis for this application.

Embodiments of the present disclosure provide an adherent lubricant, the adherent lubricant comprising one or more mucoadhesive polymers that are biocompatible with the oral cavity and nasopharynx. The adherent lubricant is one or more mucoadhesive polymers and may be selected from a group comprising: hyaluronic acid (in acid or salt form), hydroxypropylmethylcellulose (HPMC), methylcellulose, chondroitin sulfate, tamarind seed polysaccharide (TSP), guar, hydroxypropyl guar (HP guar), scleroglucan poloxamer, poly(galacturonic) acid, sodium alginate, pectin, xanthan gum, xyloglucan gum, chitosan, sodium carboxymethylcellulose, polyvinyl alcohol, polyvinyl pyrrolidine, carbomer, polyacrylic acid and combinations thereof. In an embodiment of the present disclosure, the one or more mucoadhesive polymers is a combination of a low and high molecular weight sodium hyaluronate.

The mucoadhesive polymers (such as hyaluronic acid) may be modified with a mucin binding functional group such as thiol, boronic acid or vinyl sulfone to form an adherent lubricant of the formulation. For example, Ivanov et al. describe the bonding of boronic acid (Ivanov et al., Macromol. Biosci. 2011, 11, 275-284); Lopez-Jaramillo et al. Vinyl Sulfone (Lopez-Jaramillo et al., Bioconjugate Chem. 2012, 23, 4, 846-855). Verena et al. describe the formation of disulphide bonds with mucins (Verena M. Leitner, Greg F. Walker, Andreas Bernkop-Schnürch, Thiolated Polymers: Evidence for the Formation of Disulphide Bonds with Mucus Glycoproteins, European Journal of Pharmaceutics and Biopharmaceutics, Volume 56, Issue 2, 2003, Pages 207-214).

As stated above, there are multiple mucin binding groups available to choose from. Boronic acid is preferred due to its chemistry being stable at physiological pH and its reactivity towards sialic acid being strong towards neutral and alkaline pHs. A high proportion of human mucin chains terminate in negatively charged α-2,3 and α-2,6 N-acetylneuraminic acids, which are known as sialic acids. Baos S C, Phillips D B, Wildling L, McMaster T J, Berry M. Distribution of sialic acids on mucins and gels: a defense mechanism. Biophys J. 2012 Jan. 4; 102 (1): 176-84.

The formation of a boronic ester from a boronic acid and a diol is likely the strongest, reversible functional group interaction in an aqueous environment among organic compounds that can be used for the construction of molecular receptors in multiple applications, including oral mouth applications. See, for example, Yan, G.; Springsteen, G.; Deeter, S.; Wang, B. Tetrahedron 2004, 60, 11205-11209. Thus, by combining high mucoadhesive capacity with mucin binding chemistry, this will enable excellent retention of the lubricant and overcome limitations of the existing physical binding limitations in the mouth.

Without being bound to any theory, it is known that mucin, a glycoprotein found in various bodily secretions such as mucus, contains sialic acid (with saliva comprising about 2% weight of sialic acid), and a mucin binding group such as boronic acid is capable of preferential binding to sialic acid residues of mucins through the formation of a boronate ester link, allowing for selective recognition and binding for longer lasting protection, lubrication, healing, and moisturization.

Boronic acid may also be modified by adding functional groups or substituents to the boronic acid molecule that can alter the properties and reactivity of boronic acids, expanding their potential applications depending on the environment pH and ionicity. Exemplary boronic acid derivatives include but are not limited to: Arylboronic acids that have an aryl group (e.g., phenyl, naphthyl) attached to the boron atom; Alkylboronic acids that have an alkyl group (e.g., methyl, ethyl) attached to the boron atom; and the like, and combinations thereof.

Mucin binding groups such as boronic acid may also be modified by adding functional groups or substituents to the molecule that can alter their properties and reactivity expanding their potential applications. For boronic acids, exemplary boronic acid derivatives include, but not limited to: Arylboronic acids that have an aryl group (e.g., phenyl, naphthyl) attached to the boron atom; Alkylboronic acids that have an alkyl group (e.g., methyl, ethyl) attached to the boron atom; and the like, and combinations thereof.

The formulation preferably comprises an adherent polymer that comprises a mucin-binding modified hyaluronic acid. Hyaluronic acid (HA) is a naturally occurring non-sulfated glycosaminoglycan (GAG) non-protein compound that can be manufactured to produce high quality product using biofermentation processes. The polymer possesses distinct physico-chemical properties with excellent viscoelasticity, high moisture retention capacity, high biocompatibility, and hygroscopic properties. At concentrations as low as 0.1%, the polymer chains can provide high viscosity and act as effective lubricants across a variety of biological substrata. Additional properties such as osmoregulation, tissue healing, and anti-inflammatory capabilities add the positive benefits HA provides for treatment in a number of unmet medical needs. Gupta R C, Lall R, Srivastava A, Sinha A. Hyaluronic Acid: Molecular Mechanisms and Therapeutic Trajectory. Front Vet Sci. 2019 Jun. 25; 6:192. Hyaluronic acid has a molecular weight ranging from about 100,000 grams per mole to about 7 million grams per mole. Hyaluronic acid used herein may be a combination of a medium molecular weight ranging from about 100,000 grams per mole to about 500,000 grams per mole, and a high molecular weight ranging from about 1 million grams per mole to about 7 million grams per mole. Further hyaluronic acid may be made available as a metal salt, such as a sodium salt, potassium salt, calcium salt, magnesium salt, and the like, and combinations thereof. Also, all the carboxylic acid groups may be neutralized with the metal salt, or only a partial fraction of the carboxylic acid groups may be neutralized. High and low molecular weight sodium hyaluronate can be used as a high-viscosity humectant that is known to possess exceptional properties such as facilitating water retention through water binding which can be multiple times its molecular weight.

Modification of polymers such as polysaccharides are expressed in terms of degree of modification, also sometimes known as degree of substitution or degree of derivatization. The degree of modification may be expressed in terms of a percentage, or a fraction that ranges from 0 to the maximum number possible. For example, in the case of polysaccharides having 3 hydroxyl groups, the degree of substitution ranges from 0 to 3. In some embodiments, the degree of modification of hyaluronic acid with boronic acid ranges from about 1% to about 30%, and higher percentages are possible.

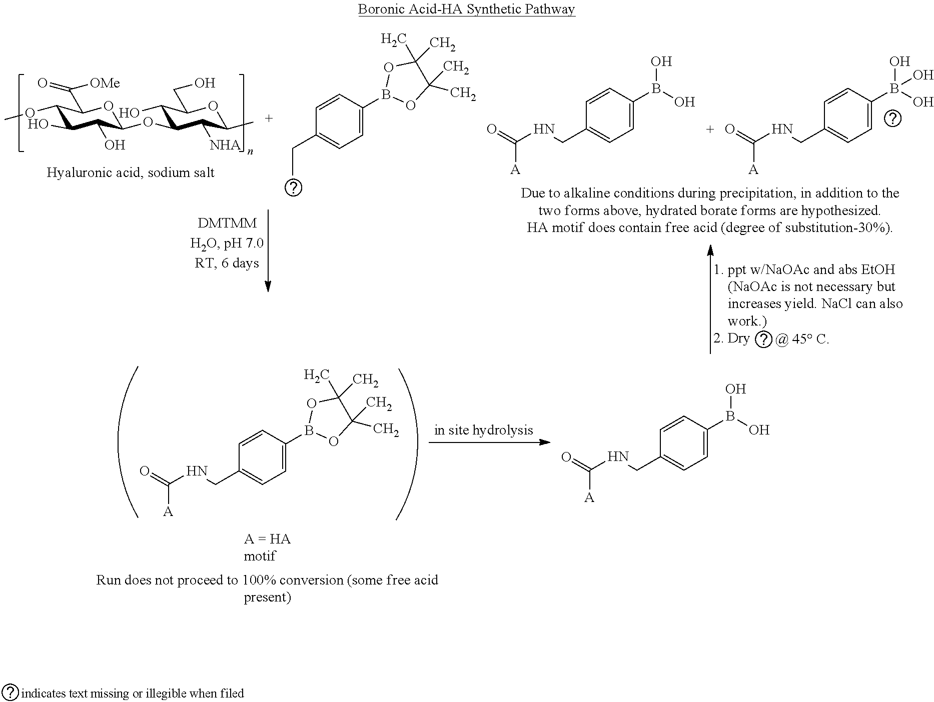

As illustrated in the table below, in tests that were conducted, the modified polymer was synthesized with boronic acid substitution percentages of about 20%+. They were then formulated in a phosphate-buffered saline solution at concentrations of 0.1%, 0.2%, and 0.3% that have demonstrated initial stability in pH ranges from 7.19-7.26 with an HPLC characterization method showing linearity R\hat{ }2 of 0.9993

| Material | Residual Starting | Substitution % |
|---|---|---|
| Lot | Boronic Acid (%) | (w/w) (estimated) |
| AX01-01 | 1.26, 1.17 | 18.3, 21.4 |
| Avg | 1.22 | 19.9 |
| AX01-04 | 2.17, 2.41 | 23.0, 23.8 |
| Avg | 2.29 | 23.4 |

To test the mucin binding potential of the modified polymer that was produced, AX01-04 (0.5% HA-PBA) was compared to a PBS control and a 0.5% unmodified hyaluronic acid comparator. Custom polydimethylsiloxane devices were adhered to mucin-functionalized glass slides and the test articles were exposed to the microfluidic channels with a PBS wash at 30 μL/min for 120 mins. Released HA or AX01-04 was detected by UV absorption. The percent release of each article was calculated and normalized to unbound samples. The area under the curve (AUC) for the percent release profiles of HA and AX01-04 were 745 and 911, respectively indicating that HA without any binding groups exhibited a 1.2-fold higher concentration in the flow-through compared to AX01-04.

Mucin was covalently linked to glass slides as previously described [1]. Briefly, 22×22 mm glass slides were cleaned by submerging slides in 2.5% (v/v) hydrochloric acid (HCl) for 3 hours, following by sonication in 2.5% (v/v) HCl for an additional 2 hours. Slides were rinsed with excess distilled water and allowed to air dry. These cleaned slides were refluxed for 2 hours with 1% (v/v) 3-aminopropyl trimethyl silane (APTES) dissolved in toluene with 2-3 drops of triethylamine. The resulting slides were rinsed with 100 mL of toluene and then with excess ethanol. Glass slides were dried under N₂ gas. Afterwards, slides were treated with 3.42% (w/v) succinic anhydride in dimethylformamide (DMF) solution overnight at 60° C. Finally, slides were functionalized with mucin by incubation with a solution of 2.5% (w/v) porcine mucin, 4% (w/v) EDCI, and 5.8% (w/v) HOBt in DMF for 4 hours at room temperature. Slides were washed with excess water and stored in dry conditions. The mucin functionalization on slides was confirmed by thiofalvin T staining.

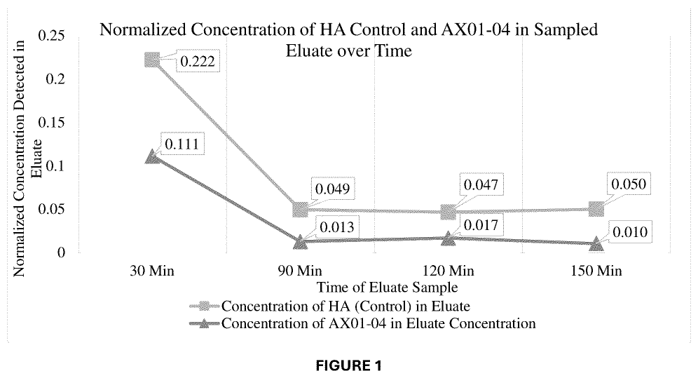

Customized polydimethylsiloxane (PDMS) devices were fabricated and were adhered to functionalized mucin glass slides, and ˜0.2 mL of sample (PBS control, 0.5% (w/v) hyaluronic acid in PBS, or 0.5% (w/v) AX01-04 in PBS) was added to the channels of the microfluidic device and incubated at 37° C. at high humidity (>80%) overnight. Any unbound sample was collected from the device by manual pipetting. Microfluidic tubing was then attached to the device, and PBS was flowed through the device at a rate of ˜ 30 mL/min using a peristaltic pump. Eluates/flow through from the device was collected every 30 minutes. Released of HA or AX01-04 was detected by monitoring UV absorption spectra from 215-280 nm. Percent release of HA or AX01-04 was calculated and normalised to unbound samples (see FIG. 1).

In summary trace amounts of both HA and AX01-04 were eluted from the device when washed with PBS. This indicates that HA exhibited a 2× higher concentration in the flow-through compared to AX01-04 at 30 minutes, 3.9× at 90 minutes. 2.8× at 120 minutes, and 4.8× at 150 minutes. This suggests that a greater proportion of AX01-04 remained bound to the mucin slide, thus less detected in the eluates, assuming equivalent binding of AX01-04 and HA to the immobilized mucin in the system. This work provides the ability to characterize the binding and percent of molecules released in the immobilized mucin experimental system. The byproduct of this work enables the incorporation of epithelial cells that express mucin into the microfluidic device.

The formulation then comprises a saliva enhancer that enhances saliva production and/or flow rate. These compounds are also sometimes known as sialagogues. An exemplary saliva enhancer useful in the formulation is xylitol. Other useful sialagogues include malic acid, ascorbic acid, pilocarpine, and the like. Other herbal extracts known to enhance saliva production and/or flow rate include aloe vera, ginger, marshmallow root, and so on.

The formulation is typically prepared in an aqueous medium at a suitable concentration such that the formulation achieves a requisite viscosity to meet the application requirements. The modification of the mucoadhesive polymers by the anchoring groups may be affected in situ in the aqueous medium. The reaction may further be enhanced using suitable catalysts or providing appropriate reaction conditions to enhance conversion. In some specific embodiments, the formulation has a viscosity that drops by at least 30% percent within about 30 seconds of being exposed to forces having a shear rate ranging from about 10 sec⁻¹ to about 1000 sec⁻¹, and the viscosity of the formulation returns to the viscosity at zero shear rate upon termination of the shear force.

The formulation further comprises a humectant such as betaine, erythritol, sorbitol, or xylitol. The formulation also comprises other ingredients that assist in the maintenance of proper hydration and electrolyte balance, such as taurine. The unique combination of these ingredients provides deep moisturizing relief at the tissue level and soothes the oral mucosa. The formulation does not contain drying and irritating agents, such as alcohol, sodium lauryl sulfate (SLS), or other sulfates, is non-oily and does not contain milk or egg derivatives. It is formulated to have a mild flavor that can help with compliance. The formulation is essentially alcohol-free. As used herein, the phrase “alcohol-free” means the formulations comprise less than about 1.0% alcohol by volume, preferably less than about 0.5% v/v alcohol, more preferably less than about 0.1% v/v alcohol, and even more preferably about 0% v/v.

It is also known that the boronate ester linkage is reversible and requires neutral to alkaline conditions to be effective. Thus, the formulation further includes buffering agents having sufficient buffer capacity to maintain the pH between about 6 to about 8. Suitable buffering agents that may be employed include but are not limited to, alkali metal hydroxides, carbonates, sesquicarbonates, borates, silicates, phosphates, imidazole, monosodium phosphate, trisodium phosphate, pyrophosphate salts, citric acid, sodium citrate, fumaric acid, malic acid, and the like, and mixtures thereof.

It is generally known that enhancement of efficacy of formulations for those suffering from dry mouth is through the improvement of contact between the formulations with the roof of the mouth. Thus, to enable this, in some embodiments, the formulation is made highly viscous and shear thinning during rinsing and provided with intentionally sticky or bioadhesive materials that enhances contact with the roof of the mouth.

In some embodiments, the formulation may include one or more additional and/or alternative ingredients. These additional and/or alternative ingredients may be provided to use the adhering film retained on the tissues as a sustained delivery approach to help retain the additional and/or alternative ingredients between the adherent film and surfaces of the oral cavity tissues, thereby facilitating increased bioavailability of the ingredient into the tissue and providing longer-lasting effects as compared to presenting the ingredient in a solution without an adherent lubricant. Non-limiting examples of the ingredients include compounds for freshening mouth breath, vitamins, ectoin, trehalose, chondroitin sulfate as a lubricating agent, ADP ribose to enhance healing, other health supplements, other drug actives or other compounds, including preservatives, colorants, alcohol, antiseptics, anticavity compounds (e.g., like sodium fluoride), and the like, and combinations thereof.

In some embodiments, the formulation includes a cooling agent, that is, an ingredient that imparts an oral cooling sensation. In some cases, the cooling sensation can be longer-lasting (e.g., at least about 30 minutes, and in some cases, at least about 60 minutes, and in some cases, from 30 to 60 minutes) than if the cooling agent is in an oral rinse with no high molecular weight polymer present (e.g., a cooling sensation of several minutes). As a non-limiting example, in some embodiments the cooling agent includes a compound in the class of Transient receptor potential cation channel subfamily M member 8 (TRPM8) agonists capable of being effectively retained in the oral tissue with the high molecular weight polymer and slowly absorbed through the mucus membrane and thereby providing long-lasting cooling. In some such embodiments, the TRPM8 agonist is provided in a concentration ranging from about 0.0001% to about 2% Wt/Vol, and in some cases, more preferably 0.4% Wt/Vol. Exemplary cooling agents include one or more of menthol, menth menthyl lactate (MML), menthol acetyl, menthyl glycerin acetal (MGA; e.g., Frescolat® menthyl glycerin acetal), and the like, and combinations thereof.

In some embodiments, the formulation includes a warming agent, that is, an ingredient that imparts an oral warming sensation. As a non-limiting example, in some embodiments, the warming agent includes a compound in the class of transient receptor potential cation channel subfamily V member (TRPV) agonists, or transient receptor potential ankyrin, member (TRPA) agonists. Non-limiting examples of such TRPV or TRPA agonists include one or more of capsaicin, piperine, vanillyl butyl ether, vanillyl ethyl ether, and the like, and combinations thereof. In some embodiments, the TRPV or TRPA agonist is provided in a concentration range of from about 0.0001% to about 2% Wt/Vol. In some embodiments, the warming agent can be co-delivered with one or more of the cooling agents listed above.

Some embodiments of the formulation may further include a sweetener. In some cases, the inclusion of a sweetener can facilitate masking of the undesirable taste of other ingredients, such as caffeine. Non-limiting examples of sweeteners include one or more of xylitol, stevia extract, monk fruit concentrate, coconut sugar, oat sugar, thaumatin, pentadin, brazzein, agave, honey, and the like, and combinations thereof. In some embodiments, the sweetener is provided in a concentration range of from about 0.001% to about 15% Wt/Vol and can include a combination of more than one type of sweetener.

Some embodiments of the formulation include natural or artificial flavoring agents, e.g., to help mask the undesirable taste of some energy supplements. In some embodiments, the flavoring agent can also advantageously serve as an antimicrobial agent to thereby prevent the growth of bacteria and yeast. Non-limiting examples of such flavoring agents include one or more of vanillin, wintergreen, peppermint oil, orange oil, spearmint oil, lemon oil, licorice, cardamom, ginger, cinnamon, clove, fennel, caraway, grapefruit, camphor, sassafras, natural and artificial cherry flavor, artificial champagne flavor, natural vanilla extract, ethylene vanillin, coffee extract, chocolate extract, artificial chocolate flavoring, cocoa extract, and the like, and combinations thereof. In some embodiments, the flavoring agent can be botanical extracts (KESITLANT Plant, Drummondville, Canada) produced using a fermentation process using naturally occurring symbiosis of bacteria and yeast.

Some embodiments contain short-chain peptides shown to reduce wound and tissue healing in epithelial membranes and stimulate growth hormones. Non-limiting examples include BPC-157, TB-500, CJC-1295, Ipamorelin, semiglutide, and other GLP-1 agonists.

In some embodiments, the formulation includes excipients that provide a fast transfer of medication or fluids across cellular membranes. Examples of excipients include propylene glycol, sodium lauryl sulphate, polyethylene glycol, DMSO, or water-insoluble materials such as hydrogenated vegetable oil.

In some embodiments, the formulation is enhanced by a nebulizer that produces a fine mist or a fine spray with droplets in the range of 2-4 μm to aid in the coverage of the spray to the oral cavity or nasopharynx. The nebulizer can be produced with a venturi that introduces the flow of air, gas, or oxygen to the medication source.

In some embodiments, the flavoring agent includes spearmint oil in Tween 20 in a 1:5 to 1:6 weight ratio added to an aqueous volume of the oral composition in a ratio of about 1:10. For example, in some cases, the oral rinse composition includes about 0.6 gm of spearmint oil added to about 3 gm Tween 20, which in turn, is added to 33 gm of water. Such compositions desirably provide a clear solution of the formulation.

In some embodiments, the flavoring agent includes grapefruit oil and peppermint oil in Tween 20 in weight ratios of about 1:2.5:46. For example, in some cases, the formulation includes about 0.13 gm of grapefruit oil and 0.33 gm of peppermint oil added to about 5.97 gm Tween 20, which in turn, is added to 33 gm of water. Such compositions desirably provide a clear solution of the formulation.

Suitable natural extracts that can be used in the formulation include those selected from the group consisting of antioxidants, immune stimulants, and combinations thereof. Such extracts contain a wide variety of phytochemicals such as polyphenols, flavanols, proanthocyanains, and other phytochemicals that are known to contribute to healing of inflammation in the oral cavity, promote healthy gum tissue, inhibit the growth of bacteria, as well as provide other beneficial effects.

Useful antioxidants may include citrus seed extract, such as grapefruit seed extract, bitter orange extract, orange extract, lemon extract, lime extract, olive leaf extract, and mixtures thereof; or olive leaf extract. Additional antioxidants that can be included in the composition include, for example, but are not limited to, co-enzyme Q-10, pine bark extract, vitamin A, vitamin C, vitamin E, zinc, and combinations thereof.

Suitable immune stimulants useful in the in the formulations disclosed herein include Echinacea (flower and root), Goldenseal, Hawthorne Berry, Myrrh, Rosehips, Lomatium dissectum, Astragalus root, Licorice root, and mixtures thereof.

The formulations may also include baking soda to clean teeth and tone gums. When used in conjunction with hydrogen peroxide, the baking soda can also help remove tooth discoloration and control tartar buildup. Further, the formulations can include salt such as sodium chloride, potassium chloride, and the like, and combinations thereof to help disinfect oral lesions. Salt may also contribute to the removal of stains and cleaning of gums. The formulation can be configured as a paste for application to the dental surfaces in the mouth.

In some instances, to aid in the complete dispersion of the ingredients in the formulation and throughout the oral area during use, nonionic surfactants can be included. Suitable non-ionic surfactants include poly(oxyethylene)-poly(oxypropylene) block copolymers (also known as poloxamers), condensates of ethylene oxide with polymers of propylene oxide, and amphoteric agents such as quaternized imidazole derivatives. The non-ionic surfactant can be individually added to the composition, or it can be added via a base composition, including the surfactant.

Suitable opacifiers, such as, but not limited to, titanium dioxide, cerium oxide (CeO2), tin oxide (SnO2), zirconium dioxide (ZrO2), and the like may be employed. Suitable pigment or colorant may be included, such as, but not limited to, a dye, an aluminum lake, a caramel colorant based upon iron oxide, and the like or a mixture thereof.

In some instances, the formulation is provided as a viscous solution that is delivered in a thick form factor at the time of entering the mouth, but the viscosity rapidly reduces as it comes in contact with bodily fluids. In another embodiment, a viscosity change occurs as a phase change in the gel-like solution in response to body temperature. In another embodiment, a viscosity change occurs as a phase change as bodily fluids precipitate the carrier upon delivery into the body. Further, the reduction of viscosity, whether by bodily fluids or exposure to body temperature, can be timed to provide a soothing sensation to the mouth by the suitable release of soothing agents.

The formulation can be provided as an oral rinse composition. Such compositions are used to rinse the mouth by swirling the formulation in the mouth for a certain period of time, for example, for 5 seconds, 10 seconds, 20 seconds, 30 seconds, 60 seconds, and so on, and then it is spit out. Thus, in one embodiment, an oral rinse composition comprising the formulation of the examples disclosed herein as described herein is provided.

In some instances, the oral rinse composition is mixed with a lidocaine rinse composition, such that a combined rinse can be applied simultaneously to alleviate and/or provide suitable therapy. In some cases, the rinse composition is applied into the nasopharynx to soothe the back of the throat and nasal passages.

In another embodiment, the formulation is made available as a gel or a wax, which is then delivered to the mouth through a suitable applicator. In such instances, the gel or wax may be formulated to release the formulation slowly over a period of time as it comes in contact with bodily fluids, such as saliva, or in response to exposure to body temperature. In another embodiment, the gel or wax degrades in response to exposure to body fluids or body temperature. The mechanical presence of the gel or wax in the mouth also stimulates the production of the patient's own saliva if possible. Thus, in another embodiment, a wax comprising the formulation as described herein is provided. In yet another embodiment, a slow-release device comprising the formulation as described herein is provided. The rate of release of the formulation from the device may be suitably tailored for a constant release to a fast release, depending on the requirement. Tailoring of device characteristics are well known to one skilled in the art.

In one form as a wax structure, the material can be applied directly on the teeth or other oral appliances in the patient's mouth. For dry mouth sufferers, the application of wax can provide a smooth intra-oral surface to the tongue and gums in locations of the mouth where irritation occurs. This irritation can be caused by dental appliances, roughened dental surfaces, swollen or infected gums or periodontal regions, or mucosal surfaces previously irritated and exacerbated by the dry mouth condition.

In another form of the wax structure, the material can be shaped and configured to fit between the gums and teeth of the patient for both a slow release of the formulation of the in a solution, and a smooth surface in an irritated portion of the mouth. The wax can be shaped like thin sheets of material from 1 mm to 3 mm in thickness and lengths of 1 cm to 5 cm. In another embodiment, the wax structure can be configured as cylindrically rounded structures with outer diameters of 2 mm to 1 cm and lengths of 1 cm to 5 cm and similar in size to rolled gauze. In another embodiment, the wax structure is configured with multiple wells and cavities that are filled with the formulation of the solution. Once applied in the mouth or attached to the teeth or dental appliance, the solution is directly released from the multiple wells or cavities.

In another embodiment, a tray that fits on the lower jaw or upper jaw can be configured with multiple wells or cavities filled with the formulation of the solution. In practice, either a lower or upper tray can be applied to the patient's mouth for dry mouth relief.

In another embodiment, the trays can be configured as a night guard in the mouth with multiple wells or cavities filled with formulation of the solution. The night guard is placed into the mouth and fits onto the teeth of the patient. The night guard configuration would then release the solution over time while the patient wears the device

In another embodiment, the gel is constructed with a bioadhesive material that allows the gel to be placed onto the gums, teeth, or roof of the mouth for a sustained period of time while releasing the formulation of the solution.

The formulation may also be made available on sponges, an applicator or “lollipop-like” configuration, or swabs so that it can be applied onto the surface of the mouth, for example the roof of the mouth. Suitable effervescent compounds or foaming agents may be included in such formulations to enhance coverage over a greater surface area during application.

In alternate embodiments, the formulation can be made available as chewing gum or ingestible compositions, which may include drops, gels, gum strips, tablets, and the like. In a specific embodiment, the formulation is made available on a suitable surface such as a nipple or at the end of a stick that provides an attraction to users (e.g., children) to use it. In further embodiments, the formulation may be made available as inhalable compositions, which may include sprays, nebulizers, and the like. Such inhalants can be used to deliver the formulation through the mouth or the nasal region. When delivered in this manner, the nasopharynx region can also be treated, for example, to treat mucus build up at the back of the throat.

In another embodiment, the formulation can be configured as a nasal wash aspirate to coat the nasopharynx and back of the throat. This can be done in combination or separately from the oral application of the formulation. The formulation can be provided in a dissolvable pouch so that it can be ingested as a film. Alternately, it can be provided in a non-dissolvable pouch wherein the pouch is a resealable or a disposable pouch, so that the formulation is delivered from the pouch, and then suitably disposed or reused accordingly. Such disposable pouches lend themselves to single-use pouches that can be used to deliver the formulation for single use situations. Such pouches provide the convenience of carrying the formulations with ease.

As stated herein, in some embodiments, the oral composition comprises an aqueous solution that includes a high molecular weight polymer and the anchoring or mucin binding group. In some embodiments, the oral composition can consist essentially of an aqueous solution having xylitol, the anchoring group, high molecular weight hyaluronic acid, sodium citrate and citric acid, calcium chloride, sodium chloride, magnesium chloride, and/or monk fruit extract at a pH of 6.0 to 6.8. Other embodiments of the oral rinse composition can further include other ingredients that synergistically promote forming the viscous and tribological films such as: combinations of different molecular species of polymers, micro- and nano-emulsions, colloidal suspensions, pH, temperature, ion sensitive polymers, or metal ion (e.g., boron) crosslinking polymer systems.

In another aspect, a method of preparing the formulation that comprises a mucoadhesive polymer and an anchoring group is provided. Any of the above-described embodiments of the formulation can be prepared according to the methods disclosed herein. The method comprises a step of providing the mucoadhesive polymer and the anchoring group in an aqueous medium. As a non-limiting example, in some embodiments, about 0.1 grams of a 1 million grams per mole molecular weight hyaluronic acid and 0.05-0.1 grams of the anchoring group are dissolved in water. In some embodiments, the water is heated above room temperature (e.g., to about 70-80° C.) to facilitate rapid dissolution.

Further embodiments include a step of adding a pH buffering agent, such as citrate, phosphate or Tris buffer. As a non-limiting example, citrate buffer comprises about 0.6 grams of sodium citrate and about 0.5 to 0.02 grams of citric acid in about 100 ml. Further, the formulations may also include emulsions or vesicle formats.

The methods may further include, without limitation, adding any one or more of the following ingredients: a surfactant, a penetration enhancer, an excipient to enhance cellular penetration, essential vitamins, a cooling agent, a warming agent, a sweetener, monovalent and/or divalent salts, a flavoring agent, or combinations thereof. In some cases, a flavoring agent is prepared as a separate solution which is then combined with the formulation.

The formulation and methods outlined herein may include one or more active ingredients for the delivery of drug or to provide for long lasting drug delivery. These additional active ingredients would provide a therapeutic such as anti-inflammatory, antibiotic, antifungal, and/or pain reduction.

### Formulation

TABLE 2

| | | Concentration |
|---|---|---|
| | Concentration | Component |
| Formulation 1 | (% Wt. Vol) | Range (% Wt/Vol) |
| Aqua (water) | 84% | 80-95% |
| Buffer Sodium Borate | 0.6% | 0.3-0.7% |
| Citric Acid | 0.3% | 0.02-0.4% |
| Lauryl Glucoside | 0.2% | 0.1-0.5% |
| Mucin Binding Hyaluronic Acid | 0.1% | 0.01-0.3% % |
| Poloxamer 407 | 0.2% | 0.1-0.5% |
| Saliva Enhancer Sweetener Xylitol | 4% | 2-10% |
| Propylene Glycol | 10% | 3-10% |
| Flavoring | 0.5% | 0.1-1% |
| Sodium Benzoate | 0.3% | 0.1-0.5% |
| Sodium Saccharin | 0.05% | 0.01-0.1% |

TABLE 3

| | Concentration | Concentration Component |
|---|---|---|
| Formulation 2 | (% Wt. Vol) | Range (% Wt/Vol) |
| Buffer Sodium | 0.53% | |
| Buffer Citrate Citric Acid | 0.053% | |
| Buffer Polyox WSK | 0.1%-5% | 0.4% |
| N-12K Caffeine | 0.05%-2% | 0.2% |
| Energy Xylitol | 0.4%-0.8% | 0.8% |
| Sweetener Ozarka Qs | To 100% Water | |
| | Solution | |
| Peppermint Flavoring | 0.01%-0.2% | 0.12% |
| Orange Oil | 0.01%-0.2% | 0.12% |
| Frescolat Oil | 0.05%-2% | 0.4% |
| Menthone Glycerin Acetal | 1%-5% | 2.4% |
| Polysorbate | | |

TABLE 18

| | Concentration | Concentration Component |
|---|---|---|
| Formulation 17 | (% Wt. Vol) | Range (% Wt/Vol) |
| Buffer Sodium | 0.53% | |
| Buffer Citrate Citric Acid | 0.053% | |
| Buffer Polyox WSK | 0.1%-5% | 0.4% |
| N-12K Caffeine | 0.05%-2% | 0.6% |
| Energy Xylitol | 0.1%-2% | 1.6% |
| Sweetener Ozarka Qs | To 100% Water | |
| | Solution | |
| Monk Fruit Sweetener | 0.1%-15% | 5.6% |
| Grapefruit Flavoring | 0.01%-1% | 0.18% |
| Menthone Oil | 0.05%-2% | 0.4% |
| Glycerin Acetal Polysorbate | 1%-5% | 2.4% |

TABLE 19

| | Concentration | Concentration Component |
|---|---|---|
| Formulation 18 | (% Wt. Vol) | Range (% Wt/Vol) |
| Buffer Sodium | 0.53% | |
| Buffer Citrate Citric Acid | 0.053% | |
| Buffer Polyox WSK | 0.1%-5% | 0.4% |
| N-12K Caffeine | 0.05%-2% | 0.6% |
| Energy Xylitol | 1%-15% | 3% |
| Sweetener Ozarka Qs | To 100% Water | |
| | Solution | |
| Lemongrass Flavoring | 1%-20% | 1.8% |
| Cinnamon Extract Flavoring | 1%-20% | 1.8% |
| Cardamom Fermented Extract | 0/01%-1% | 0.08% |
| Menthone Oil | 0.05%-2% | 0.4% |
| Glycerin Acetal Polysorbate | 1%-5% | 1.5% |

Those skilled in the pertinent arts to which this application relates will appreciate that other and further additions, deletions, substitutions and modifications may be made to the described embodiments.

While only certain features have been illustrated and described herein, many modifications and changes will occur to those skilled in the art. It is, therefore, to be understood that the appended claims are intended to cover all such modifications and changes as fall within the true spirit of the disclosure.

We claim:

1. A formulation of a mucoadhesive polymer having an anchoring group constructed and arranged to bind and anchor to the saliva in the mouth to enhance the duration of the existing saliva in the mouth.

2. A formulation for treating dry mouth in humans comprising:
a mucoadhesive polymer;
an anchoring group; and
a saliva enhancer.

3. The formulation of claim 2, wherein the anchoring group comprises boronic acid.

4. The formulation of claim 2, wherein the mucoadhesive polymer has a molecular weight ranging from about 100,000 grams per mole to about 7 million grams per mole.

5. The formulation of claim 4, wherein the mucoadhesive polymer molecular weight ranges from about 1 million grams per mole to about 3 million grams per mole.

6. The formulation of claim 4, wherein a degree of derivatization of the mucoadhesive polymer by the anchoring group ranges from about 1% to about 30%.

7. The formulation of claim 2, wherein the mucoadhesive polymer is hyaluronic acid.

8. The formulation of claim 2, wherein the saliva enhancer is xylitol.

9. The formulation of claim 2, further includes water.

10. The formulation of claim 9, wherein the mucoadhesive polymer modified by said anchoring group is present at a concentration ranging from about 0.01% wt./vol to about 10% wt./vol.

11. The formulation of claim 9, wherein said saliva enhancer is present at a concentration ranging from about 0.001% wt./vol to about 2% wt./vol.

12. The formulation of claim 9, wherein said formulation has a room temperature viscosity as measured by using a Brookfield viscometer at 12 rpm ranging from about 1 centipoise to about 100 centipoise.

13. The formulation of claim 9, further comprising buffering agents to maintain a pH of the formulation ranges from about 6 to about 8.

14. The formulation of claim 2, further comprising at least one of a surfactant, a skin and mucosal penetration enhancer, a stabilizer, a cooling agent, a warming agent or a flavoring agent, a vitamin, an antioxidant, or a combination thereof.

15. An oral rinse composition comprising the formulation of claim 2.

16. The formulation of claim 2, further including an active ingredient that is at least one of an anti-inflammatory, an antibiotic, an antifungal, pain reduction, or combinations thereof.

17. An oral rinse composition, comprising:
a. an aqueous medium;
b. xylitol;
c. sodium citrate and citric acid;
d. menthone glycerin acetal;
e. polysorbate 20;
f. spearmint oil;
g. peppermint oil;
h. vitamin E;
i. monk fruit extract;
j. a hyaluronic acid having a molecular weight of about 1 million grams per mole modified polymer with 20% boronic acid; and a buffering agent to maintain a pH of 6 to 6.8.

18. A method of making an oral rinse composition, comprising dissolving a mucoadhesive polymer modified with an anchoring group and xylitol in water.

19. The method of claim 18, wherein the oral rinse composition further comprises an aqueous dispersion and wherein the aqueous dispersion is prepared by mixing a flavoring agent and a surfactant in water to form a uniform aqueous dispersion.

20. The method of claim 18, further comprising adding a buffering agent to maintain a pH of the oral rinse composition in a range from about 6.5 to about 8.

21. The method of claim 18, further comprising sterilizing the oral rinse composition.

22. The method of claim 18, further comprising filtering the oral rinse composition.

23. A delivery system comprising the formulation of claim 17 and at least one active pharmaceutical ingredient.

24. The delivery system of claim 23, wherein the active ingredient comprises at least one of an anti-inflammatory, an antibiotic, an antifungal, pain reduction, or combinations thereof.

25. A method of making the dry mouth formulation of claim 2, comprising dissolving the mucoadhesive polymer modified with the anchoring group and xylitol in water.
